(19) United States
(12) Patent Application Publication
(10) Pub. No.: US 2008/0200745 A1
(43) Pub. Date: Aug. 21, 2008

(54) Method For Producing Propene From 2-Butene And Isobutene-Rich Feeding Flows

(75) Inventors: Marcus Sigl, Mannheim (DE); Markus Schubert, Ludwigshafen (DE); Jurgen Stephan, Mannheim (DE); Frank Poplow, Ludwigshafen (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, LUDWIGSHAFEN (DE)

(21) Appl. No.: 11/817,250

(22) PCT Filed: Feb. 24, 2006
(86) PCT No.: PCT/EP06/60275

§ 371 Date: Sep. 27, 2007

(30) Foreign Application Priority Data
Feb. 28, 2005 (DE) 10-2005-009-665-4

(51) Int. Cl. C07C 2/00

(52) U.S. Cl. 585/643

(57) ABSTRACT

Process for preparing
a stream consisting essentially of ethene (stream C₂⁼),
a stream consisting essentially of propene (stream C₃⁼),
a stream consisting essentially of isobutene and possibly other olefins having 4 carbon atoms (stream C₄⁼) and
a stream consisting essentially of 2-methyl-2-butene and possibly 2-butene (stream C₅⁼),
and, if appropriate, a stream which comprises other hydrocarbons (stream Cₓ) and may consist of a plurality of separate substreams,
   which comprises a metathesis step in which a C₄-hydrocarbon stream comprising at least 15% by weight of 2-butenes, at least 5% by weight of isobutene and not more than 5% by weight of 1-butene (feed stream) is brought into contact with ethene in the presence of a customary metathesis catalyst and the resulting hydrocarbon stream is fractionated to give the streams C₂⁼, C₃⁼, C₄⁼ and, if appropriate, Cₓ and all or part of the stream C₅⁼ is recirculated to the metathesis step.

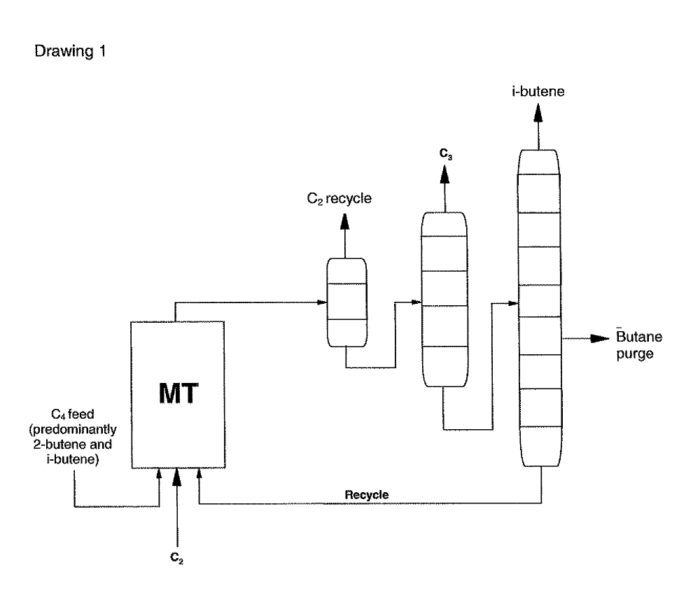

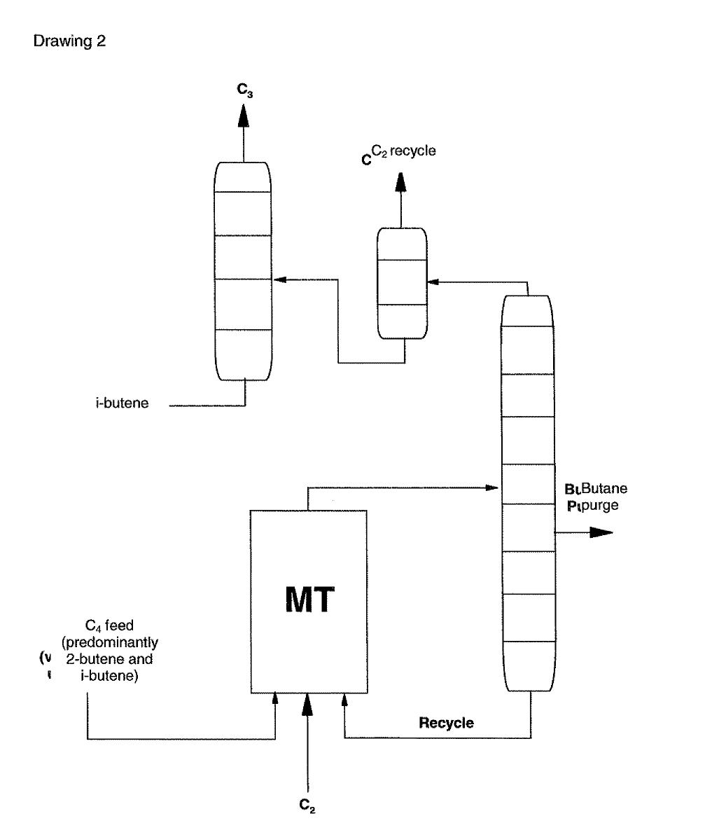

The present invention relates to a single-stage process for preparing propene by ethenolysis of a 2-butene- and isobutene-comprising stream.

Ethenolysis of 2-butenes is a long-established method of preparing propene (e.g. Weis-sermehl, K., Arpe, H.-J., Chapt. 3.4 “Olefin-Metathese” in “Industrielle Organische Chemie”, 4th Edition, VCH, Weinheim 1994).

It is also known that not only pure 2-butene but also 2-butene-rich C4 fractions can be used as feedstock for the preparation of propene. DE 101,18,634, EP 0,832,867, EP 1,069,101, EP 0,936,206, DE 197,46,040 and EP 1,134,271, for example, disclose single-stage and multistage metathesis processes for reaction of a mixture of linear butenes known as raffinate II for coproduction of propene and hexene or propene and 1-butene. Raffinate II is a 1-butene- and 2-butene-comprising C₄ stream which has previously gone through an isobutene removal step, so that the residual isobutene content is less than 3%. Very similar single-stage processes, but with substantially lower 1-butene contents, are described in EP 0,742,195, EP 0,742,234, EP 1,110,933 and EP 1,110,934. Common to all these processes is the fact that isobutene has to be substantially separated off beforehand in a separate step before the remaining 2-butene-rich stream can pass to the metathesis step.

DE 102,14,442 describes a process for the direct reaction of an isobutene-rich stream in the metathesis. However, a disadvantage here is the two-stage nature of the process in which C₅-olefins are firstly formed as intermediates in a first reactor and then have to be worked up by ethenolysis in a second metathesis reactor.

Various types of catalysts are known in principle to those skilled in the art for a heterogeneously catalyzed metathesis. For the temperature range up to about 120° C., the use of supported Re₂O₇ or Re(CO)₁₀ catalysts is customary (Mol, J. C., Chapt. 4.12.2 “Alkene Metathesis” in “Handbook of Heterogeneous Catalysis”, Eds. Ertl, G., Knözinger, H., Weitkamp, J., VCH, Weinheim 1997). A person skilled in the art will use Al₂O₃, preferably gamma-Al₂O₃, as support for this purpose. At somewhat higher temperatures up to 400° C., it is possible, according to the literature, to employ catalysts based on MoO₃, CoO—MoO₃, MoS₂, Mo(CO)₆ or various supported Mo complexes, and at even higher temperatures up to 540° C., systems based on WO₃, WS₂, W(CO)ᵣ or supported W complexes can also be used (Mol, J. C., Chapt. 4.12.2 “Alkene Metathesis” in “Handbook of Heterogeneous Catalysis”, Eds. Ertl, G., Knözinger, H., Weitkamp, J., VCH, Weinheim 1997; Weissermehl, K., Arpe, H.-J., Chapt. 3.4 “Olefin-Metathese” in “Industrielle Organische Chemie”, 4th Edition, VCH, Weinheim 1994; Heckelsberg, L. F., Banks, R. L., Bailey, G C., Ind. Eng. Chem. Prod. Res. Develop. 8 (1969), 259-261). A person skilled in the art will give preference to SiO₂— and/or Al₂O₃-supported tungsten oxides for the metathesis at relatively high temperatures.

It was an object of the present invention to develop a very simple process which makes possible a very economical preparation of propene from a 2-butene-rich and isobutene-comprising C4 stream which is nevertheless low in 1-butene by means of an ethenolysis reaction.

We have accordingly found a process for preparing
a stream consisting essentially of ethene (stream C₂⁼),
a stream consisting essentially of propene (stream C₃⁼),
a stream consisting essentially of isobutene and possibly other olefins having 4 carbon atoms (stream C₄⁼) and
a stream consisting essentially of 2-methyl-2-butene and possibly 2-butene (stream C₅⁼),
and, if appropriate, a stream which comprises other hydrocarbons (stream Cₓ) and may consist of a plurality of separate substreams,
   which comprises a metathesis step in which a C₄-hydrocarbon stream comprising at least 15% by weight of 2-butenes, at least 5% by weight of isobutene and not more than 5% by weight of 1-butene (feed stream) is brought into contact with ethene in the presence of a customary metathesis catalyst and the resulting hydrocarbon stream is fractionated to give the streams C₂⁼, C₃⁼, C₄⁼ and, if appropriate, Cₓ and all or part of the stream C₅⁼ is recirculated to the metathesis step.

The feed stream preferably comprises
from 15 to 94% by weight, particularly preferably from 25 to 85% by weight, of 2-butenes
from 5 to 60% by weight, particularly preferably from 8 to 40% by weight, very particularly preferably from 9 to 18% by weight, of isobutene
from 0 to 5% by weight, particularly preferably less than 2% by weight, of 1-butene

As further significant constituent, the feed stream often comprises butanes, usually in amounts of from 1 to 60% by weight.

In general, the feed stream is prepared by one of the two processes comprising steps Ia and IIa or steps Ib and IIb.

In the case of the process comprising the steps Ia and IIa, the procedure comprises
Ia) in step Ia, subjecting naphtha or other hydrocarbon compounds to a steam cracking or FCC process and taking off a C₄-olefin mixture comprising isobutene, 2-butene and butadienes and possibly butynes and possibly 1-butene from the resulting stream and
IIa) preparing a C₄-hydrocarbon stream (raffinate I) consisting essentially of isobutene, 2-butenes and possibly butanes and possibly 1-butene from the C₄-olefin mixture formed in step Ia by hydrogenating the butadienes and butynes to butenes or butanes by means of selective hydrogenation or removing the butadienes and butynes by extractive distillation to such an extent that the 1,3-butadiene content is not more than 1000 ppm by weight.

In the case of the process comprising the steps Ib and IIb, the procedure comprises
Ib) in step Ib, preparing a C₄-olefin mixture comprising isobutene, 2-butenes and butadienes and possibly, butynes possibly 1-butene and possibly butanes from a butane-comprising hydrocarbon stream by dehydrogenation and subsequent purification,
IIb) preparing a C₄-hydrocarbon stream (raffinate I) consisting essentially of isobutene, 2-butenes and possibly butanes and possibly 1-butene from the C₄-olefin mixture formed in step Ib by hydrogenating the butadienes and butynes to butenes or butanes by means of selective hydrogenation or removing the butadienes and butynes by extractive distillation to such an extent that the 1,3-butadiene content is not more than 1000 ppm by weight.

In the generally known FCC process (cf. Ullmann's Encyclopedia of Industrial Chemistry, Wiley-VCH Verlag GmbH, Weinheim, Germany, Sixth Edition, 2000 Electronic Release, Chapter Oil Refining, 3.2. Catalytic Cracking) the respective hydrocarbon is vaporized and is in the gas phase brought into contact with a catalyst at a temperature from 450 to 500° C. The particulate catalyst is fluidized by the hydrocarbon stream conveyed in countercurrent. Catalysts employed are usually synthetic crystalline zeolites.

In the likewise generally known steam cracking process (cf. A. Chauvel, G. Lefebvre: Petrochemical Processes, 1 Synthesis—Gas Derivatives and Major Hydrocarbons, 1989 Editions Technip 27 Rue Ginoux 75737 Paris, France, Chapter 2), the hydrocarbon is mixed with steam and, depending on the residence time, heated to temperatures of from 700 to 1200° C. in tube reactors and then rapidly cooled and separated by distillation into individual fractions.

The substep of selective hydrogenation is preferably carried out in the liquid phase over a metal selected from the group consisting of nickel, palladium and platinum on a support, preferably palladium on aluminum oxide, at a temperature of from 20 to 200° C., a pressure of from 1 to 50 bar, a space velocity of from 0.5 to 30 m³ of fresh feed per m³ of catalyst per hour and a ration of recycle to feed stream of from 0 to 30 and a molar ratio of hydrogen to diolefins of from 0.5 to 50.

Preference is given to carry out a butadiene extraction of the C₄-olefin mixture using a butadiene-selective solvent selected from the class of polar aprotic solvents such as acetone, furfural, acetonitrile, dimethylacetamide, dimethylformamide and N-methylpyrrolidone.

Particular preference is given to a combination of extractive distillation and selective hydrogenation in which, if the 1,3-butadiene content of the C₄-olefin mixture obtained in step Ia or Ib is 5% by weight or more, the 1,3-butadiene content is reduced to a content in the range from 1000 ppm by weight to 5% by weight by means of extractive distillation and the 1,3-butadiene content is subsequently reduced further to 1000 ppm by weight or less by means of selective hydrogenation.

Subsequent to one of the steps Ia, IIa, Ib or IIb, the 1-butene content of the feed stream is, if necessary, reduced to the above-defined content by converting most of the 1-butene in the C₄-olefin mixture or raffinate I into 2-butene by hydroisomerization or preferably converting most it selectively into valeraldehyde in a hydroformylation reaction and separating off the valeraldehyde.

The hydroformylation reaction can generally be carried out in the manner which is customary and known to a person skilled in the art. A good overview with numerous further references may be found, for example, in M. Belier et al., Journal of Molecular Catalysis A, 104, 1995, pages 17 to 85, or in Ullmann's Encyclopedia of Industrial Chemistry, 6^{th} edition, 2000 electronic release, Chapter “ALDEHYDES, ALIPHATIC AND ARALIPHATIC-Saturated Aldehydes”.

In the hydroformylation reaction, preference is given to preparing valeraldehyde (n-pentanal) from 1-butene in the presence of a transition metal catalyst with addition of synthesis gas (CO:H₂ of from 3:1 to 1:3, preferably from 1.5:1 to 1:1.15).

Rhodium complexes with phosphorus-containing ligands are generally used as catalysts for the hydroformylation reaction. The phosphorus-containing ligands are typically monophosphanes or diphosphanes, preferably triarylphosphanes, particularly preferably triphenylphosphane. The hydroformylation reaction is usually carried out at temperatures of from 50 to 150° C., preferably from 70 to 120° C., and pressures of from 5 to 50 bar, preferably from 10 to 30 bar.

If necessary, the isobutene content of the feed stream or the raffinate I can be reduced to the desired values in each case by known methods.

There are in principle four fundamentally different possible ways of achieving this: a) removal by distillation, b) removal by means of etherification/extraction, c) direct polymerization to form polyisobutene and d) oligomerization.

Distillation (method a) takes place in an apparatus suitable for this purpose, e.g. a bubble cap tray column, a column packed with random packing, a column packed with audit packing or a dividing wall column. The distillation column preferably has from 20 to 80 theoretical plates. The reflux ratio is generally from 5 to 50. The distillation is generally carried out at a pressure of from 5 to 20 bar.

Due to the low boiling point of isobutene and 1-butene compared to 2-butenes and n-butane, the stream taken off overhead will comprise mainly isobutene and 1-butene, while the stream taken off at the bottom will comprise mainly 2-butenes and n-butane. The content of low boilers (isobutene and 1-butene) in the stream taken off at the bottom is less than 40%, preferably less than 30% and particularly preferably 5-20%. The content of high boilers (2-butenes and n-butane) in the stream taken off overhead is less than 40%, preferably less than 30% and particularly preferably from 5 to 20%.

In the case of method b), the usual procedure is to bring the feed stream or raffinate I into contact with an alkyl alcohol, preferably a C₁-C₄-alkyl alcohol, and a customary catalyst for the formation of alkyl tert-butyl ether and to separate off the alkyl tert-butyl ether formed. Particularly preferred alcohols are methanol and butanols.

The etherification is preferably carried out in the presence of an acid ion exchanger in a three-stage reactor cascade in which flow occurs from the top downward through flooded fixed-bed catalysts, with the reactor inlet temperature being from 0 to 60° C., preferably from 10 to 50° C., the outlet temperature being from 25 to 85° C., preferably from 35 to 75° C., the pressure being from 2 to 50 bar, preferably from 3 to 20 bar, and the ratio of alcohol to isobutene being from 0.8 to 2.0, preferably from 1.0 to 1.5.

In the case of method c), the usual procedure is to bring the feed stream or raffinate I into contact with a customary catalyst for the polymerization of isobutene and to separate off the polyisobutene formed from the remaining feed stream or raffinate. As catalyst, preference is given to using a homogeneous or heterogeneous catalyst from the class of Brönsted or Lewis acids. The catalyst is preferably boron trifluoride or aluminum trichloride. The oligomerization of isobutene (method d) is carried out at temperatures in the range from 50 to 150° C. and pressures in the range from 10 to 40 bar with catalysis by protons. Preference is given to using sulfuric acid or acid ion exchangers as catalysts. Main products are dimers, a lesser amount of trimers and also high oligomers as by-products.

Before the feed stream is introduced into the metathesis, additional feed purification is normally necessary to decrease the traces of oxygen compounds and dienes, in particular allenes, to an amount which is tolerable in the metathesis. The total content of oxygen compounds, for example water, acetone or ethanol, after the purification step should be less than 100 ppm by weight, preferably less than 50 ppm by weight, particularly preferably less than 10 ppm by weight. The content of diolefins should be less than 300 ppm by weight, preferably less than 150 ppm by weight, particularly preferably less than 100 ppm by weight. Allenes, 1,2-butadiene and propadiene in particular in the feed are serious poisons for metathesis catalysts and should be present in an amount of less than 15 ppm by weight, preferably less than 10 ppm by weight, very particularly preferably less than 5 ppm by weight. The purification of the C₄ stream comprises one or more steps and can also comprise pressure swing adsorption processes, but preference is given to at least one adsorptive process. The purification is preferably carried out directly before the metathesis step. The purification step can optionally also comprise a selective hydrogenation to remove remaining traces of diolefins which have not been completely hydrogenated in the first selective hydrogenation step or have been formed afresh in later process steps, for example in isomerization. The purification stage preferably comprises at least one adsorbent bed based on an aluminum oxide or a molecular sieve to remove oxygen compounds. Particularly preference is given to an embodiment in which at least two adsorbent beds based on an aluminum oxide or molecular sieve are present and are alternately in the adsorption mode and the regeneration mode. Preferred adsorbents are 13× molecular sieves and high-surface-area gamma-aluminum oxides.

The feed stream and ethene and part or all of the stream C₅⁼ are brought together in the metathesis step, with from 0.5 to 2 mol, preferably from 0.9 to 1.2 mol, of ethene being used per mole of 2-butenes present in the feed stream.

For the metathesis, two different types of catalyst are in principle possible: a) rhenium-comprising catalysts which are operated at temperatures in the range from 0 to 150° C., preferably in the range from 35 to 110° C., and b) W-comprising, Re-free catalysts which are operated in the gas phase at temperatures of from 200 to 600° C., preferably from 220 to 450° C.

The Re comprising catalysts preferably comprise at least 1% by weight of Re in oxidic form on a support which comprises at least 75% by weight of a high-surface-area aluminum oxide, very particularly preferably gamma-aluminum oxide. Particular preference is given to catalysts which have an Re content in the range from 5 to 12% by weight and are supported on pure gamma-Al₂O₃. The catalysts can further comprise dopants, for example oxides of Nb, Ta, Zr, Ti, Fe, Mn, Si, Mo, W, phosphate or sulfate, to increase the activity. The catalysts have surface areas of at least 50 m²/g, preferably at least 100 m²/g, and a pore volume of at least 0.3 ml/g, preferably at least 0.4 ml/g. Suitable Re-containing catalysts are, for example, described in DE-A-102004009804, DE-A-102004009805 or DE-A-102004009803.

Suitable W-comprising, Re-free catalysts preferably comprise at least 3% by weight of W, at least partly in oxidic form, on a support selected from the group consisting of aluminum oxide, aluminosilicates, zeolites and, preferably, SiO₂. The catalysts preferably have a surface area of at least 50 m²/g and a pore volume of at least 0.3 ml/g, particularly preferably at least 0.5 ml/g. The activity or isomerization activity can be altered by means of suitable dopants, for example alkali metal compounds and alkaline earth metal compounds, TiO₂, ZrO₂, HfO₂, or compounds or elements from the group consisting of Ag, Sb, Mn, W, Mo, Zn, Si.

A person skilled in the art will know that all types of metathesis catalysts have to be regenerated oxidatively at regular intervals. For this purpose, a set-up comprising fixed beds and at least two reactors of which at least one reactor is always in the regeneration mode, or, as an alternative, a moving bed process in which deactivated catalyst is discharged and regenerated externally can be employed.

The hydrocarbon stream formed in the metathesis step is generally fractionated by generally known distillation methods, if appropriate in a plurality of stages, to give the streams C₂⁼, C₃⁼, C₄⁼ and, if appropriate, Cₓ.

In an alternative embodiment, the fractionation can be carried out by firstly separating off ethene overhead from the product stream comprising unreacted ethene, 2-butene, isobutene and possibly 1-butene and also the products 2-methyl-2-butene and propene and possibly further C₅- and C₆-olefins in the first work-up step and recirculating it to the metathesis. In a second step, propene is separated off overhead in “polymer-grade” purity. In the last column, an overhead stream having an isobutene content of at least 70% by weight, preferably more than 80% by weight is finally separated off and can be passed to further use. The bottoms comprising mainly 2-butene, 2-methyl-2-butene and possibly other C₅- and C₆-olefins are returned to the metathesis reaction. To avoid an accumulation of butane, it is usually also necessary to take off a C₄ purge stream in the third column. Higher olefins having 7 and more carbon atoms (C₇₊) may also be formed as a result of secondary reactions such as double bond isomerization or dimerization. To prevent accumulation of these components, an additional C₇₊ purge stream can optionally be taken off. This purge stream C₇₊ is preferably taken off at the bottom of the column on which the butane purge is also carried out. This embodiment is shown in drawing 1 in the interests of clarity.

In an alternative embodiment, the fractionation can be carried out by firstly separating off ethene together with propene and isobutene overhead from the product stream comprising unreacted ethene, 2-butene, isobutene and possibly 1-butene and also the products 2-methyl-2-butene and propene and possibly further C₅- and C₆-olefins in the first work-up step and recirculating the bottoms comprising predominantly 2-butene, 2-methyl-2-butene and possibly further C₅- and C₆-olefins to the metathesis. In a second step, ethene is separated off overhead and fed back to the metathesis step. In the third column, propene in polymer-grade purity is finally separated off overhead and bottoms having an isobutene content of at least 70% by weight, preferably more than 80% by weight, are obtained and can be passed to a further use. To avoid accumulation of butane, it is also necessary to take off a C₄ purge stream from the first column. This embodiment is shown in drawing 2 in the interests of clarity.

Polymer-grade propene generally meets the following specification:

| | | |
|---|---|---|
| Propene | >99.5% | by weight |
| Propane | <5000 | ppm by weight |
| Methane | <200 | ppm by weight |
| Ethane | <300 | ppm by weight |
| Ethene | <30 | ppm by weight |
| Acetylene | <1 | ppm by weight |
| Water | <10 | ppm by weight |
| Sulfur | <2 | ppm by weight |
| C₃H₄ | <5 | ppm by weight |
| Total C4 | <10 | ppm by weight |
| H₂ | <10 | ppm by weight |
| N₂ | <20 | ppm by weight |
| O₂ | <2 | ppm by weight |
| CO | <3 | ppm by weight |
| CO₂ | <5 | ppm by weight |
| Oxygen | <4 | ppm by weight |

### Compounds

The fractionation of the hydrocarbon stream formed in the metathesis step is advantageously carried out so that the stream Cₓ comprises a substream comprising mainly C₆⁼-olefins.

In the process of the invention, a 2-butene-rich feed is fed to a metathesis step in order to convert the 2-butene into the higher-value monomer propene. An important aspect of the process of the invention is that although isobutene and any residual 1-butene present in the feed stream likewise partly react to form higher olefins (C₅ and C₆), these are at least partly returned to the metathesis after the fractional distillation of the product stream, so that they are mostly converted into isobutene and little or no net conversion occurs. Losses thus occur mostly through purge streams by means of which the accumulation of compounds such as n-butane or i-butane or any high-boiling by-products (C₇⁺) in the feed stream is prevented.

In the external balance, higher olefins are preferably formed in a maximum amount of 20 mol % based on the propene, particularly preferably in an amount of not more than 10 mol % and very particularly preferably in an amount of not more than 5 mol %.

For this reason, the part of the stream C₅⁼ which is recirculated to the metathesis step is made at least so large that the amount of isobutene present in the stream C₄⁼ is at least 80% of the amount of isobutene present in the feed stream.

Furthermore, wherein the stream C₅ is therefore preferably recirculated to the metathesis in at least such an amount that not more than 0.2 mol, preferably not more than 0.1 mol, very particularly preferably not more than 0.05 mol, of the olefins having 5 or 6 carbon atoms formed in the metathesis step per mole of propene present in the stream C₃⁼ is not recirculated to the metathesis step.

The stream C₂⁼ can advantageously be utilized by recirculating the stream C₂⁼ to the metathesis step or combing it with an ethene fraction obtained in the steam cracking or FCC process of step Ia.

### EXAMPLES

### Example 1

A 2-butene- and isobutene-rich stream is reacted in the liquid phase over a fixed-bed catalyst, viz. 10% by weight of Re₂O₇ on gamma-Al₂O₃. Ethene, consisting of fresh ethene and recycle ethene, is added in a stoichiometric amount based on the C₄ content. To compensate for deactivation of the catalyst, the temperature is gradually increased from 35 to 120° C. during the reaction over a period of from one to two weeks. The reaction pressure is 35 bar. The catalyst then has to be regenerated oxidatively. During this time, a parallel reactor (A/B mode of operation) can take over production. In the first work-up step, C₂ is firstly separated off overhead from the product stream comprising unreacted ethene, 2-butene, isobutene and possibly 1-butene and also the products 2-methyl-2-butene and propene and possibly further C₆- and C₆-olefins and is recirculated to the metathesis. In a second step, propene is separated off overhead in polymer grade purity. In the last column, an overhead stream having an isobutene content of at least 70%, preferably more than 80%, is finally separated off and can be passed to a further use. The bottoms comprising mainly 2-butene, 2-methyl-2-butene and possibly other C₅- and C₆-olefins are returned to the metathesis reaction. In addition, a C₄ purge stream has to be taken off from the third column to avoid accumulation of butane.

### Example 2

A 2-butene- and isobutene-rich stream is reacted in the gas phase over a fixed-bed catalyst, viz. 10% by weight of WO₃ on SiO₂. Ethene, consisting of fresh ethene and recycle ethene, is added in a stoichiometric amount relative to the C₄ content. To compensate for deactivation of the catalyst, the temperature is gradually increased from 230 to 430° C. during the reaction over a period of a few weeks. The catalyst then has to be regenerated oxidatively. During this time, a parallel reactor (A/B mode of operation) can take over production. In the first work-up step, C₂ together with C₃ and isobutene are firstly separated off overhead from the product stream comprising unreacted ethene, 2-butene, isobutene and possibly 1-butene and also the products 2-methyl-2-butene and propene and possibly further C₅- and C₆-olefins, and the bottoms comprising predominantly 2-butene, 2-methyl-2-butene and possibly further C₅- and C₆-olefins are recirculated to the metathesis. In a second step, ethene is separated off overhead and recirculated to the metathesis step. In the third column, propene is finally separated off overhead in polymer-grade purity and bottoms having an isobutene content of at least 70%, preferably more than 80%, are obtained and can be passed to a further use. In addition, a C₄ purge stream has to be taken off from the first column to avoid accumulation of butane.

1.-17. (canceled)

18. A process for preparing propene by ethenolysis of a C₄-stream containing 2-butene and isobutene, but being poor in 1-butene, comprising
a metathesis step in which a C₄-hydrocarbon stream comprising at least 15% by weight of 2-butenes, at least 5% by weight of isobutene and not more than 5% by weight of 1-butene (feed stream) is brought into contact with ethene in the presence of a metathesis catalyst and the resulting hydrocarbon stream is fractionated to give a stream consisting essentially of propene (stream C₃⁼), and further fractionated in
a stream consisting essentially of ethene (stream C₂⁼),
a stream consisting essentially of isobutene and possibly other olefins having 4 carbon atoms (stream C₄⁼) and
a stream consisting essentially of 2-methyl-2-butene and possibly 2-butene (stream C₅⁼),
and optionally a stream which comprises other hydrocarbons (stream Cₓ) and optionally contains a plurality of separate substreams,
and wherein all or part of the stream C₅⁼ is recirculated to the metathesis step.

19. The process according to claim 18, wherein the part of the stream C₅⁼ recirculated to the metathesis step is at least so large that the amount of isobutene present in the stream C₄⁼ is at least 80% of the amount of isobutene present in the feed stream.

20. The process according to claim 18, wherein the stream C₅⁼ is recirculated to the metathesis in at least such an amount that not more than 0.2 mol of the olefins having 5 or 6 carbon atoms formed in the metathesis step per mole of propene present in the stream C₃⁼ is not recirculated to the metathesis step.

21. The process according to claim 18, wherein the feed stream comprises from 15 to 94% by weight of 2-butenes, from 5 to 60% by weight of isobutene, from 1 to 60% by weight of butanes and from 0 to 5% by weight of 1-butene.

22. The process according to claim 18, wherein the stream Cₓ comprises a substream which consists essentially mainly of C₆⁼-olefins.

23. The process according to claim 18, wherein from 0.5 to 2 mol of ethene is used per mole of 2-butenes present in the feed stream.

24. The process according claim 18, wherein the feed stream is prepared by
Ia) in step Ia, subjecting naphtha or other hydrocarbon compounds to a steam cracking or FCC process and taking off a C₄-olefin mixture comprising isobutene, 2-butene and butadienes and possibly butynes and possibly 1-butene from the resulting stream and
IIa) preparing a C₄-hydrocarbon stream (raffinate I) consisting essentially of isobutene, 2-butenes and possibly butanes and possibly 1-butene from the C₄-olefin mixture formed in step Ia by hydrogenating the butadienes and butynes to butenes or butanes by means of selective hydrogenation or removing the butadienes and butynes by extractive distillation to such an extent that the 1,3-butadiene content is not more than 1000 ppm by weight.

25. The process according to claim 18, wherein the feed stream is prepared by
Ib) in step Ib, preparing a C₄-olefin mixture comprising isobutene, 2-butenes and butadienes and possibly 1-butene, possibly butynes and possibly butanes from a butane-comprising hydrocarbon stream by dehydrogenation and subsequent purification,
IIb) preparing a C₄-hydrocarbon stream (raffinate I) consisting essentially of isobutene, 2-butenes and possibly butanes and possibly 1-butene from the C₄-olefin mixture formed in step Ib by hydrogenating the butadienes and butynes to butenes or butanes by means of selective hydrogenation or removing the butadienes and butynes by extractive distillation to such an extent that the 1,3-butadiene content is not more than 1000 ppm by weight.

26. The process according to claim 24, wherein, if the 1,3-butadiene content of the C₄-olefin mixture obtained in step Ia or Ib is 5% by weight or more, the 1,3-butadiene content is reduced to a content in the range from 1,000 ppm by weight to 5% by weight by means of extractive distillation and the 1,3-butadiene content is subsequently reduced further to 1,000 ppm by weight or less by means of selective hydrogenation.

27. The process as claimed in claim 18, which comprises a single-stage or multistage purification of the feed stream suitable for removing oxygen compounds or traces of diolefins and acetylenes in which the feed stream is passed through an adsorber bed comprising at least one adsorbent from the group consisting of molecular sieves, aluminosilicates and aluminum oxide.

28. The process according to claim 18, wherein the purified feed stream comprises from 10 to 300 ppm by weight of 1,3-butadiene, less than 100 ppm by weight of oxygen compounds and less than 15 ppm by weight of 1,2-dienes.

29. The process according to claim 18, wherein a metathesis reaction is carried out in the gas phase at temperatures of from 200 to 600° C. and pressures of from 1 to 40 bar over a catalyst comprising tungsten oxide in the metathesis step.

30. The process according to claim 18, wherein a metathesis reaction is carried out at temperatures of from 30 to 150° C. and pressures of from 1 to 50 bar over a rhenium-containing catalyst which comprises at least 1% by weight of Re in oxidic form on a support material comprising at least 75% by weight of aluminum oxide in the metathesis step.

31. The process according to claim 18, wherein the metathesis catalyst has a surface area of more than 50 m²/g, a pore volume of more than 0.3 ml/g and a bulk density of more than 300 g/l.

32. The process according to claim 18, wherein the stream C₃⁼ is worked up so that polymer-grade propene is formed.

33. The process according to claim 18, wherein the stream C₂⁼ is recirculated to the metathesis step.

34. The process according to claim 24, wherein the stream C₂⁼ is combined with an ethene fraction obtained in the steam cracking or FCC process as described in step Ia.
